(19) United States
(12) Patent Application Publication
(10) Pub. No.: US 2025/0339326 A1
(43) Pub. Date: Nov. 6, 2025

(54) HIGH OXYGEN LEVEL ENCLOSED AREAS AND METHODS OF PHYSICAL EXERTION THEREIN AND THEREWITH

(71) Applicant: AGAPE FITNESS, LLC, Greenville, SC (US)

(72) Inventor: Brian G. Morin, Greenville, SC (US)

(21) Appl. No.: 18/655,346

(22) Filed: May 6, 2024

(51) Int. Cl. A61G 10/02; A61M 16/10; A61M 16/12; A61M 16/20; A63B 71/04

(52) U.S. Cl. CPC A61G 10/026; A61M 16/101; A61M 16/12; A61M 16/201; A63B 71/04; A61M 2016/1025; A61M 2202/0208; A61M 2202/0266; A61M 2205/056

(57) ABSTRACT

An enhanced process for individuals to undertake physical activities in an oxygen-rich environment, specifically within an enclosed area. Such a process utilizes a unique device to increase oxygen levels within such a location through either higher partial pressure oxygen concentration therein or reduced partial pressures of other gases within such an environment (such as nitrogen, for example) which thereby allows for greater amounts of oxygen. Such a definitive increase in oxygen concentrations within such an enclosed space allows for more intense exercise, whether typical exertion (weightlifting, stationary biking, treadmill running/walking, utilizing exercise apparatus, even sexual intercourse, as non-limiting examples) to permit increases in calorie burning, increased muscle strength in shorter times, and enhanced physical fitness results as well. A device generating such increased oxygen concentrations with partial pressure modifications (oxygen increase, nitrogen decrease), as well as an enclosed room therewith, are also encompassed herein.

### FIELD OF THE DISCLOSURE

An enhanced process for individuals to undertake physical activities in an oxygen-rich environment, specifically within an enclosed area. Such a process utilizes a unique device to increase oxygen levels within such a location through either higher partial pressure oxygen concentration therein or reduced partial pressures of other gases within such an environment (such as nitrogen, for example) which thereby allows for greater amounts of oxygen. Such a definitive increase in oxygen concentrations within such an enclosed space allows for more intense exercise, whether typical exertion (weightlifting, stationary biking, treadmill running/walking, utilizing exercise apparatus, even sexual intercourse, as non-limiting examples) to permit increases in calorie burning, increased muscle strength in shorter times, and enhanced physical fitness results as well. A device generating such increased oxygen concentrations with partial pressure modifications (oxygen increase, nitrogen decrease), as well as an enclosed room therewith, are also encompassed herein.

### BACKGROUND OF THE TECHNOLOGY

Physical exertion and exercise have long been undertaken by individuals to improve a number of personal physiological conditions. For instance, such oxygen intake during physical exertion procedures have been utilized to increase muscular strength and size, to reduce weight through burning increased caloric levels, to improve physical stamina, and possibly to slow aging effects. Oxygen masks, for instance, particularly attached through a hose to a gaseous source (hyperoxic gases) have been developed and utilized in relation to certain training exercise regimens, whether in terms of providing increased oxygen levels or to monitor certain conditions in that manner. Problematically, however, such mask usage has proven quite cumbersome and limiting as freedom of movement and reliability of attachment during strenuous activities is questionable, at best. Intense activities such as weightlifting, boxing, basketball, football or other intense activities are impossible while wearing such an oxygen-enhancing mask and bladder system. Such hyperoxic breathing systems may be beneficial, but, again, such deficiencies limit the versatility and capability of such mask-based devices and procedures.

Other sleep or exercise enhancements have been proposed utilizing simulated changes in altitude within an enclosed space. Such suggested methods utilize oxygen concentrators and carbon dioxide scrubbers to modify air concentrations to match the partial pressure of oxygen to that experienced at certain altitudes, whether from a high to a low or vice-versa result. Such methods, however, do not involve any atmospheric partial pressures of oxygen above that experienced at a sea level equivalency, rather solely in terms of altitude-related simulated oxygen and carbon dioxide levels, ostensibly to “thin” the subject atmosphere to simulate actual training at high altitude levels. As it is, it is well understood that athletes, for example, have trained in high altitude regions (for instance, Colorado Springs, Mexico City, and the like) in order to acclimate themselves to such stressed environments as well as to benefit from such body-altering conditions. Such training is known to limit the oxygen available during exercise and, over time, to physiologically cause an increase in an athlete's red blood cells. Such a red blood cell increase can improve performance when competitive conditions are at a higher oxygen content than those experienced while training. This prior methodology thus is drastically limited to such enclosed spaces with modified oxygen levels related to high altitude concentrations, not increases to the effect that total partial pressures are elevated.

Exercise with oxygen therapy (EWOT) is a well-known technique that uses masks connected to bags/tanks of oxygen concentrated gas mixtures. The benefits of EWOT are well-documented, particularly increased oxygen circulation and blood flow within as well as increased production of adenosine triphosphate (ATP). Such physiological changes are known to improve immunity, boost energy, improve cardiovascular health, improve vision, allow for faster recovery after workouts, reduce inflammation, and accelerate weight loss. However, the utilization of masks significantly inhibits one's range of motion during EWOT, particularly prohibiting an athlete's capability of practicing core competitive activities as mentioned above. There is thus a need to provide athletic workouts, particularly with full range of motion, with the benefits of EWOT results (and full physiological effects of a partial pressure of oxygen higher than that experienced at sea level on earth) without the restrictions and difficulties of wearing a mask to do so.

As such, even though there are certain benefits accorded individuals through the utilization of oxygen masks and simulated altitude concentrations and effects as previously disclosed, such possibilities are unfortunately noticeably limited in terms of comfort as well as overall effect obtained therefrom during actual usage. There are seemingly desirable results that may yet to be determined through oxygen-enriched enclosures; to date, however, such possibilities have yet to be explored. The difficulties in generating an oxygen-enriched atmosphere within an enclosed area, particularly, are simply lacking in the pertinent art. Certainly, iron lungs and other like individualized chambers (hyperbaric, for instance) have been provided for medical and therapeutic purposes in the past; however, such specific types of structures are, analogous to oxygen masks, cumbersome for the user and lack versatility and capability, for that matter, to allow freedom of movement for physical exertion and other like activities (let alone for multiple persons to undertake exercise, etc., simultaneously). As a result, there are no disclosures providing beneficial oxygen enrichment within enclosed areas for the undertaking of individualized, couple, or group physical exertion activities therein. The heretofore unexplored capabilities of such procedures leave open the potential for effects and results that have likewise been untested. To date, there is lacking a reliable, unencumbered, oxygen-enriched exercise platform throughout the industry.

### ADVANTAGES AND SUMMARY OF THE DISCLOSURE

A distinct advantage of the disclosed processes of individual, couple, or group physical exertion activities within an enclosed room is the ability to undertake such procedures within an oxygen-enriched atmosphere. Another advantage of this disclosed process is the ability to avoid the utilization of masks, chambers, or other individual implements for oxygen delivery during such physical exertion activities, thereby allowing individuals greater versatility of movement and overall exercise capabilities and regimens in comparison with mask-based alternatives. Yet another distinct advantage is the ability to undertake such enclosed room physical exertion activities at a higher oxygen level thereby resulting in more intense workouts in terms of human physiology. Still another distinct advantage of such a disclosure is the unique provision of at least one sealed room for multiple individual presences therein with oxygen partial-pressure changes permitted in relation to on-demand controls or oxygen sensor-based automated modifications.

Accordingly, this disclosure encompasses a room which is filled and maintained with the partial pressure of oxygen gas greater than 21.2 kPa (which is the standard oxygen content at sea level). A more preferred partial pressure might be as high as 25 kPa, or more preferably 30 kPa, or 35 kPa or even 40 kPa or most preferably 50 kPa. Of course, the room could be pressurized above one atmosphere total pressure, which could allow for a partial pressure of above 100 kPa, or 200 kPa. However, the physiological effects of exercising in very high pressures are unknown, as well as a flammability and explosiveness increase with very high oxygen concentrations, and thus the maximum would be suggested to be less than 500 kPa, preferably less than 250 kPa, or most preferably less than 100 kPa or even less than 75 kPa partial pressure of oxygen. A method utilizing such a room by at least one individual, said method involving the undertaking of physical exertion activity while present therein said room with an oxygen enriched atmosphere of at least said partial pressure level at sea level. The utilization of said room within a method of physical exertion by at least two people individually or in tandem is also encompassed herein. Furthermore, a device for controlled delivery of an oxygen-enriched atmosphere exhibiting said minimum partial pressure thereof is further encompassed, whether controlled by manual capability or through an automated operation in relation to an oxygen sensor, is encompassed herein as well.

The minimum system for delivering and controlling the oxygen in an exercise room suitable for exercise comprises:
an oxygen concentrator, such as one utilizing a pressure swing adsorption process which may produce a stream of oxygen rich gas (up to 95%) and also produce a stream of nitrogen rich gas (which may alternatively also include a tank to store concentrated oxygen and a tank to store the concentrated nitrogen that is also generated by the process);
a valve to control the flow of oxygen from the oxygen concentrator to the subject room (which, alternatively, may also include a valve to control the flow of nitrogen from the oxygen concentrator to the subject room);
a distribution system, to distribute oxygen to various parts of the room (that may alternatively include a system to mix the oxygen with the other gases in the room);
the room in which the exercise or activity will occur;
a door or other means for entering and exiting the room, such door will provide adequate sealing to prevent excessive leakage of oxygen or entrance of outside air; and
a sensor in the room to determine the oxygen level (preferably, this may include multiple sensors).

Also present may be a controller with a feedback loop working in tandem with the sensor to determine the oxygen level within the closed and sealed room, in order to then open or close the valve to allow ingress of oxygen (or nitrogen) to increase or decrease the oxygen level until the desired oxygen level is achieved, and further capable of monitoring the oxygen content and opening and closing valves for the ingress of oxygen-rich or nitrogen-rich gases to increase or decrease the oxygen level. This operation allows the attainment and maintenance of the desired oxygen level while activity occurs inside the sealed, oxygen-enriched exercise chamber. The minimum process for providing an oxygen-rich environment for exercise or other physical exertion activity would include the steps of:
providing an oxygen-enhanced exercise chamber consisting of:
   an oxygen concentrator such as a pressure swing adsorption device,
   a sealed room that is large enough for the physical activity and has a means for ingress and egress,
   an oxygen sensor capable of measuring the oxygen concentration in the room,
   a means for setting a desired oxygen concentration in the room,
   a valve for allowing flow of oxygen rich gas from the oxygen-enrichment means to the sealed room,
utilizing the setting means to set the oxygen concentration to a desired concentration higher than 21.2 kPa,
providing a controller capable of controlling the steps mentioned herein, and
utilizing the sensor to measure the oxygen concentration in the room, and comparing it to the desired concentration.

Initially, such oxygen concentration should be equal to that of atmospheric air, and as such lower than 21.2 kPa, and lower than the desired concentration,
utilizing the controller to open the valve to allow the flow of oxygen-rich gas into the room for a set time period, close the valve to stop the flow of oxygen,
measuring the concentration of oxygen in the room and comparing it to the set value,
wherein, if the oxygen concentration in the room is equal or greater than the set value, take no action and measure the concentration of oxygen in the room either continuously or at set intervals,
wherein, if the oxygen concentration in the room is less than the set value, opens the valve to allow oxygen-rich gas to enter the room for a set period, then close the valve to stop the flow of oxygen.

In this way, the oxygen concentration in the room is raised to the desired elevated level, and then, if it drops lower than the desired level, more oxygen is introduced into the room to increase it until it again achieves the desired level. Because the room will be immersed in atmospheric air, the oxygen level will slowly leak out from imperfect seals, or from the means for ingress and egress from the room, and additional oxygen will need to be introduced to bring it again to the desired level. As an alternative, a step may be added if the oxygen concentration is greater than the desired concentration in which the valve to a tank with nitrogen-rich gas is opened, allowing the nitrogen-rich gas to flow into the room to lower the oxygen concentration. Since the oxygen concentrator creates both oxygen-rich and nitrogen-rich gas streams, this is a way that would be desirable to, say, cycle between two different desirable levels, both of which are above 21.2 kPa of oxygen.

The room disclosed herein may be of any size and type as long as it may be enclosed and include a door (preferably with seals to ensure the retention of the oxygen-enriched atmosphere generated therein) for ingress and egress thereto. Such a room may be a standalone structure or may be erected as part of a larger building, such as within a gymnasium, home, spa, office, and the like. The dimensions of such a room will ostensibly depend upon the number of individuals present therein during a physical exertion activity, particularly with sufficient distance therebetween such individuals for comfort and convenience (if a workout activity is followed) or a suitable area for location of a bed or like article for typical intercourse activities. Additionally, however, such a room dimensions may also rely upon the facilitation of retaining desired oxygen-enrichment levels and thus the ability to prevent oxygen depletion upon increased concentrations thereof as well as, alternatively, or in addition thereto, the ability to prevent external atmospheric exposure which may dilute the oxygen levels (higher partial pressures) therein. Such dimensions thus may result in an area of from about 20 square feet to 100,000 square feet (without limitation), with a maximum area basically dependent upon the ability to retain the desired oxygen-enriched atmosphere during the entirety of the physical exertion activity (or activities) undertaken by the individual or individuals involved, with heights ranging from eight to 30 feet (or more possibly). As an extreme example, one could envision creating an indoor practice or game arena for a field game such as football or soccer. Such an arena might have a dimension of up to 3000 ft by 3000 ft, and a height of 100 ft. or more. While this would take a very large oxygen enrichment system, such an arena is possible and would be included within the scope of this invention as proving a chamber suitable for enhanced physical activity.

Entrance to the room may be through an anti-chamber which includes doors to the outside, normal atmospheric oxygen environment and also to the oxygen-enriched environment, which is situated and sealed in such a way as to prevent significant depletion of the oxygen in the oxygen enriched environment. In addition, this room can be ventilated in such a way as to allow the oxygen to be reduced for a duration to enable the person entering not to bring in too much outside air, or the person leaving not to bring out too much oxygen, which could be dangerous in an environment where such things as smoking, vaping, candles and other ignition sources are not prohibited.

The room may be constructed of different materials with the aim to reduce the potential for oxygen-caused flames by accident. To that end, external room surfaces (e.g., surfaces that are in contact with or exposed to such oxygen enrichment) should be limited to structures/materials of wood, plastic, rubber, or other non-conductive materials, including materials with antistatic constituents present therein, as well. Paints and coatings may be provided on the walls and floors thereof that are non-conductive or, as above, antistatic in nature. Such materials may include those listed under the Wikipedia article on “Fire-safe polymers,” or also “List of fire-retardant material,” any of which can be used for building materials, furniture or other surfaces. As an example, silicone rubber is well known to be very fire resistant, stable to 500 degrees Fahrenheit.

The potential for any flames occurring within the oxygen-rich environment may be significantly lowered by both reducing the flammability of exposed materials and also reducing the propensity of spark generation, as well. Thus, the use of flame retardants, flame retardant and intumescent paints, silicone or neoprene rubber surfaces, smooth movements of moving parts, and flame- and spark-resistant electronics are desirable.

To provide the necessary oxygen enrichment within the subject room, any type of oxygen source, such as, without limitation, a directly attached purified oxygen generator (within or outside the room), a container with purified oxygen storing previously generated oxygen therein, or combinations thereof. Such oxygen sources will include a suitable conduit for transfer of oxygen for partial pressure increases therein the subject room through a hose, vent, or combination thereof. Such a generator/source provides the necessary concentration of oxygen for introduction within a sealed room to, as noted throughout, increase the partial pressure of oxygen component gas within such a sealed-room environment/atmosphere. In this manner, the actual enrichment of the room's environment does not impart prior art altitude simulations, but, to the contrary, increases oxygen partial pressure levels to imitate sea level measurements with increased oxygen concentrations, thereby allowing individuals therein exposure to increased oxygen during physical exertion activities.

Examples of oxygen concentration processes include a pressure-swing-adsorption, in which a high-pressure air is run past a zeolite molecular sieve which adsorbs the nitrogen from the subject air thereby allowing concentrated oxygen to flow past.

Such oxygen sources will be operated through any number of possible actions, particularly to impart the desired partial pressure concentration increases on demand or automatically. Thus, a user may select a certain amount of oxygen enrichment concentrations within such a subject room (a dial or like control, whether manual or remotely provided) based upon a gauge or like instrument for such a purpose. Certainly, for safety purposes, a maximum amount of oxygen concentration increase may be set within the manual or remote activator. Alternatively, or possibly in addition thereto, the system may employ oxygen sensors within the subject room in order to permit analysis of such partial pressure levels therein at any time. In such a manner, a pre-set level may be programmed during a certain time period, dependent upon the number of individuals present within the subject room during such a time period, may be undertaken in relation to the subject room and oxygen source(s). Additionally, the pre-set program may also adjust the oxygen levels within the subject room during a time period (such as an initial set partial pressure level for 15 minutes of a workout or other type of physical exercise or activity, then increase further the oxygen level for another 15 minutes, and a third increase for another 15 minutes (of course, without limitation, as any programmed oxygen-enrichment process in this manner may be implemented). In any event, such an oxygen sensor (or possible array of a plurality of oxygen sensors) is attached to any type of control apparatus which allows setting the oxygen partial pressure within the subject room to a desired level via a valve that will open to allow more oxygen into the room and close when levels are at suitable or pre-set levels (thus to provide a static or substantially static amount of oxygen enrichment within the subject room, dependent potentially, at least on the strength of seals within the subject room). The system may also include a separate atmospheric air source to allow for return to standard partial pressure levels (at sea level, at least), ostensibly to ensure such a subject room may be utilized at ambient levels in addition to selected oxygen enrichment concentrations. Additionally, if not alternatively, a fan or blower may be present within the subject room to mix such oxygen from an external source with the subject room atmosphere, at least to permit a thorough mix of the introduced oxygen and the subject room atmosphere.

Thus, the subject room oxygen sensor may be utilized not only to determine a pre-set or at least desired oxygen enrichment level, but such a sensor, or possibly a separate sensor, may be employed to ensure the amount of oxygen introduced does not generate too high an oxygen concentration. In case such an oxygen level gets too high a ventilation component may be activated to introduce outside air or other low oxygen gas within the subject room (for emergency purposes or otherwise). Such low-oxygen gas may include the concentrated nitrogen that is also generated by the pressure swing adsorption process, which may be stored for this purpose. Such a ventilator component may be of any type of device, such as a simple blower with aligned with a wall vent that may open and close on demand or as necessary (such as in relation to oxygen sensor levels measuring too great an oxygen concentration) which works in tandem with the blower to introduce external air within the subject room.

The subject room may further include carbon dioxide scrubbers in order to remove excess amounts generated through individual respiration within the enclosed space thereof. Although carbon dioxide is a very small component generally within atmospheric gas mixtures (nitrogen and oxygen are by far the largest components), the potential for rather significant amounts of such a gas in relation to human respiration may create a need for such scrubbers to be utilized, particularly if the length of time of such a physical exertion activity within the subject room is extended. Such carbon dioxide scrubbers can also have a sensor and a feedback loop, in order to maintain target levels of carbon dioxide.

The subject room may also include humidifiers in order to increase the humidity of the room, which would lower the probability of static electricity accumulation and the subsequent sparks that could ignite flammable materials. The humidifiers can also have a sensor and a feedback loop in order to maintain the target levels of humidity.

The subject room described herein would thus necessarily include a certain number of items therein and externally thereto to function properly. Thus, as a broad overview and listing, such a subject room may include, without limitation, multiple sources of oxygen, multiple valves, multiple entryways and exits. There may be other typical building components present within the subject room, such as plumbing lines, sinks, water fountains, urinals and toilets. Additionally, the subject room may further include displays for viewing (televisions, etc., for instance), as well as windows that would, in one sense, allow such displays to be outside the subject room in order to minimize the presence of electronic sources (which could create spark issues) inside the subject room. Indications of “no smoking” or “no vaping”, or other such signs that may indicate that the environment is not safe for fire or sparks may also be present therein. There may also be a sound system for instruction or for music (again, preferably with the source thereof external to the subject room and sound “piped” in for safety. There may also be incorporated therein such a subject room sound passthroughs to allow instructors or coaches to be outside the room while working with exercisers in the room.

Noted above, the subject room will include some manner of ingress and egress for individuals to enter and exit as desired. Such an implement may be a standard door provided with a means to prevent excess oxygen from leaving the subject room or excess low-oxygen air from outside entering the subject room. Such means (or implements) may include a wooden, plastic, or other material door with seals along the edges thereof for sealing purposes (as noted above). Such a manner of ingress and egress may further include an airlock-type formation and structure to allow for an individual to enter an antechamber (vestibule) initially, closing the external door thereto, and then entering the subject room through a second door (or set of doors). The airlock component may be subject to a balancing of oxygen-enrichment levels prior to ingress within the subject room, or, likewise, or perhaps alternatively, after egress from the subject room, the airlock is allowed to balance to an outside oxygen level prior to exit through the external door. In any event, the important issue is that the means for ingress and egress for individuals includes a sealed component to prevent loss of enriched oxygenated air from the subject room and/or significant introduction of outside air within the subject room. An antechamber may be potentially preferred in this instance to prevent two different doors (one from the outside and one leading directly to the subject room) being opened simultaneously. A device may be employed ensuring such door opening instances are not simultaneous, but occur at different times, as well. It should be noted that such ingress and egress means may also include curtains or other soft air-blocking materials which can be pushed aside to enter. Staggered openings would be preferable if such are implemented, best assuring the subject room oxygen levels remain with high partial pressures concentrations as above, of course. Seals along the edges of such curtain, etc., would be desirable as well. Additionally, it is desirable to have at least two exits, located at opposing sides of the room. The room and safety of the individuals contained thereof could also benefit from having additional exits, such as having at least three exits, four exits, or even five exits. While there is no maximum number of exits necessary, depending on the size of the room, a practical limit would be imposed by the perimeter and surface area of the room.

Flammability is certainly an issue with elevated oxygen concentrations (many manufacturing locations, hospitals, etc., require monitoring to avoid sparks and open flames, including smoking, vaping, electrical shorts, and the like, that may ignite such increased oxygen amounts and cause unwanted fires and explosions) and thus such carefully selected surface materials is an initial manner of protecting users from possible flammability dangers with such oxygen-enriched enclosed areas. Surface materials may be non-conductive materials such as plastics (nylon, polyester or other polymeric materials well known in the art) or rubbers such as neoprene or silicone. Beyond such room construction materials, there would be a need to carefully select devices, articles, and other implements present therein as well. For example, within a gymnasium milieu, weights, or objects weighing more than 10 lbs. (and which, thus, may create sparks upon being dropped to a certain material floor) may require special coating to prevent spark generation therewith. In such situations, it may be necessary to provide metal objects (whether weights, exercise machines, and the like) with certain coatings, including, without limitation, wood, plastic, rubber or other non-conductive materials, as well as antistatic and/or non-conductive paints. Certain other objects that may be employed for physical exertion activities may require some modifications, as well. For instance, objects that move may require smoother motions utilizing rubber or plastic stops to prevent metal-to-metal contact of certain device components during workouts, etc. Additionally, weight racks may be necessarily coated with wood, plastic, rubber or other non-conductive materials, as above, too. Additional recommendations would be similar to those included in the publication, NFPA 53, Recommended Practice on Materials, Equipment, and Systems Used in Oxygen-Enriched Atmospheres, which is incorporated herein by reference.

Exercise equipment within such a subject room may include, without limitation, aerobic equipment, such as exercise bikes, treadmills, elliptical exercise equipment, rowing machines, stair climbing machines, recumbent pedaling machines, or other equipment designed to elevate the heart rate of a person when used. Exercise equipment may also include, without limitation, equipment designed for strength training, such as weight, barbells, dumbbells, resistance machines, resistance bands, kettle balls, weight machines, equipment for body-weight exercises such as mats, balls, tubes and other equipment. Again, proper coating of such machinery (particularly if metal is prevalent, and particularly metal-on-metal contact during movement) may be needed to best ensure safety with elevated oxygen concentrations within a subject room including such devices.

The subject room's internal area may thus be designed to inhibit or at least lack a propensity for spark generation, as noted above, to prevent or at least reduce the potential for flammable events related to elevated oxygen levels therein. Additionally, such a subject room may be outfitted with flammability control mechanisms including sprinkler and other fire abatement systems (preferably automatic in nature upon indication of sparks, smoke, or flames). Such protections allow for greater reliability of the overall physical exertion system within a subject room.

Such an overall system thus provides more intense workouts than comparative exercises undertaken in lower oxygen content environments. On initiation, a control apparatus will be set to the desired oxygen content level, which will be more than 21.2 kPa. The valve from the oxygen source will be opened to allow oxygen to flow into the room until the desired level is achieved. Then the valve will be closed, and the oxygen sensor will continue to monitor the oxygen level. When the level goes below the set point, the valve will again be opened to allow more oxygen to flow into the room. Anytime the oxygen is flowing into the room, the mixing apparatus will be operating to mix the gas contents of the room.

At such a point, the subject room will operate roughly like a normal exercise room (or bedroom for physical activity related to sexual intercourse, whether pleasurable, procreative, or both). The door will open to allow an individual to enter. Such an individual may then exercise as selected, engaging with the equipment or performing equipment-less exercises such as sit-ups or jumping jacks, yoga, calisthenics or other like activities. After a suitable time has passed, the individual will then exit the subject room. As alluded to above, such physical exertion activities may be undertaken by a sole individual at one time within the subject room or a plurality of individuals may undertake similar or different exercises for any desired times. Such physical exertion activities may utilize machinery and implements (weights, for instance, without limitation) and/or involve team sports or activities, including, without limitation, tennis, racquetball, football, basketball, hockey, field hockey, volleyball, badminton, squash, jai alai, lacrosse, baseball, and the like, basically sports and like activities that may be undertaken within an enclosed and sealed area that may be subject to oxygen-enriched atmospheres as described above.

### BRIEF DESCRIPTION OF THE DRAWINGS

FIG. 1 shows the components needed for a potentially preferred embodiment of a disclosed oxygen-enriched exercise facility.

FIG. 2 shows a depiction of a potential embodiment of the oxygen generating components of a disclosed room.

FIG. 3 shows a potentially preferred depiction of an exercise facility of the disclosure with certain equipment present.

### DESCRIPTION OF THE DRAWINGS AND PREFERRED EMBODIMENTS

As noted above, the unique high oxygen enriched enclosed room covers a range of different issues and subject matter. Without any limitation intended, the following descriptions present different possible embodiments related to the disclosure.

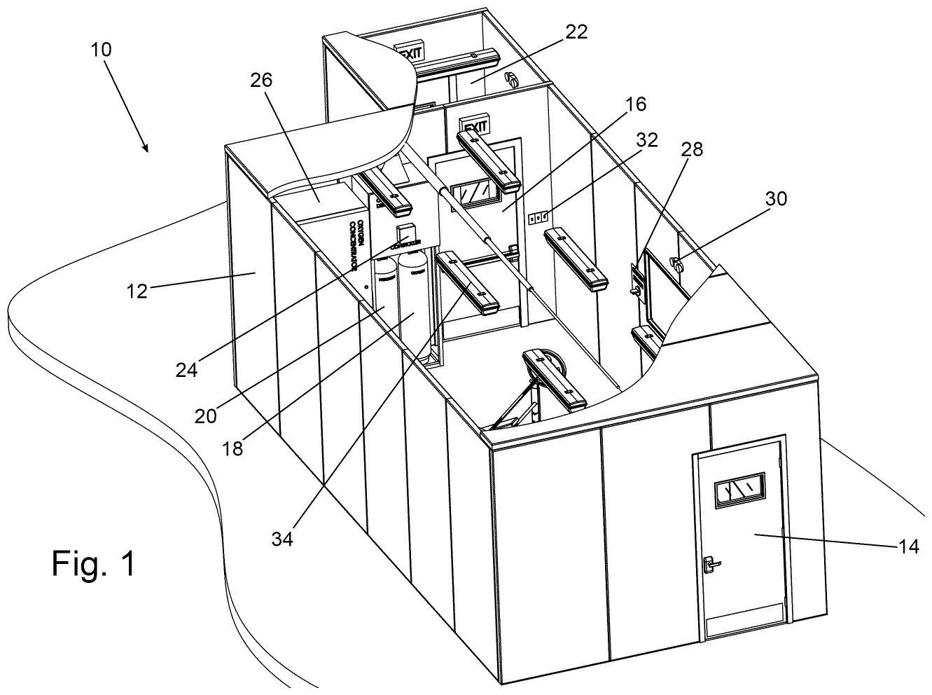

FIG. 1 of the drawings provides a partial cutaway isometric view of a potentially preferred exercise facility 10 in accordance with the disclosure. The facility 10 includes an exterior wall 12 an exterior door 14 (with another on the opposite side attached to a vestibule 22) as well as an interior door 16 allowing for exercise, etc., to commence within an interior room. Within the interior room are present two gas tanks 18, 20 of oxygen and nitrogen as well as a condenser 24 and an oxygen concentrator 26 to control the amount of oxygen introduced therein in terms of increased partial pressures thereof. An oxygen sensor 28 is present to monitor the partial pressures thereof in order to provide continued information to a user of the level of oxygen increase within the enclosed interior room, for instance. In such a manner, the ability to not only control and monitor such an oxygen level is permitted, but the ability to ensure that such a level does not exceed certain levels that may be deemed unsafe for the benefit and ultimate safety of a user thereof. Light controls 32 are provided to control overhead devices 34 as is customary within enclosed rooms. A further safety component, such as a sprinkler 30, is also provided, in case a spark creates a potential fire due to the increased presence of oxygen within such a facility 10.

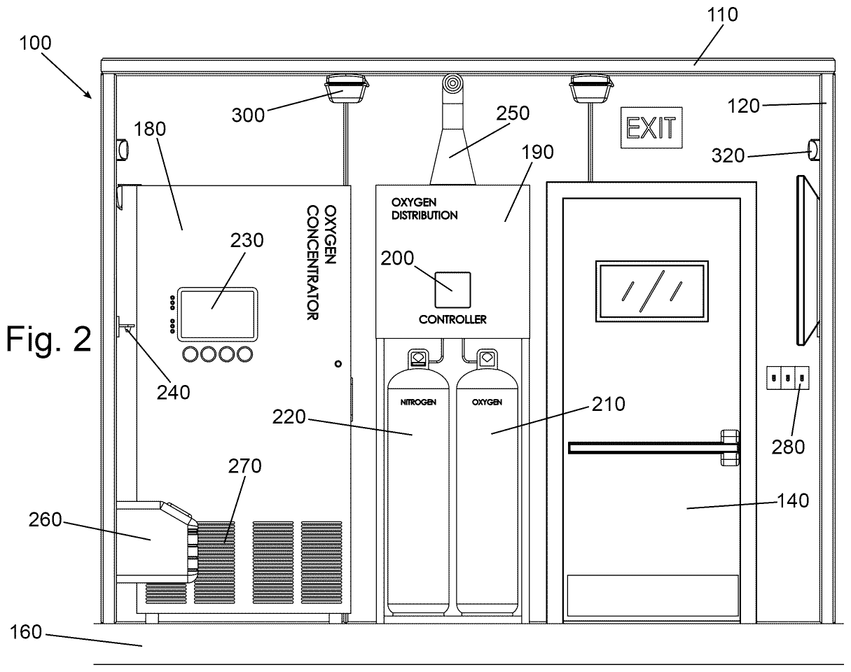

FIG. 2 shows a side view of a potentially preferred oxygen generating device set-up within a disclosed facility 100 (or room). A ceiling 110 is present as well as a floor 160 and exterior walls 120 to enclose such a facility 100, with a sealable door 140 to permit ingress and egress therein and therefrom. An oxygen concentrator 180 (26 in FIG. 1) is provided to work with the oxygen tank 210 and nitrogen tank 220 to provide the necessary increase or decrease in oxygen partial pressures within the sealable facility 100. Such a concentrator 180 and gas tanks 210, 220 are operated ostensibly through an oxygen distribution component 190 and an embedded controller 200. The oxygen concentrator 180 includes a readout screen 230 to indicate the amount of oxygen released into the facility atmosphere at any time, with the distribution controller 200 formatted to allow for such oxygen level increases or decreases on demand A vent 250 attached to the oxygen distribution component 190 allows for such oxygen release, with oxygen sensor 240 monitoring such release to ensure protections for any user as well as suitable oxygen levels are provided for maximum or at least desired exercise levels upon such increased oxygen exposure is undertaken during an athletic workout or like activity therein. Furthermore, the oxygen concentrator 180 includes a cooling component 260 and vents 280 to help control the temperature of the concentrator 180 during utilization thereof. A light switch 280 may include controls for overhead lights 300 and possibly an override for the oxygen concentrator 180 or distributor 190, if necessary. A sprinkler 320 is provided as one possible device to help upon the generation of a spark therein the facility 100 (as noted above).

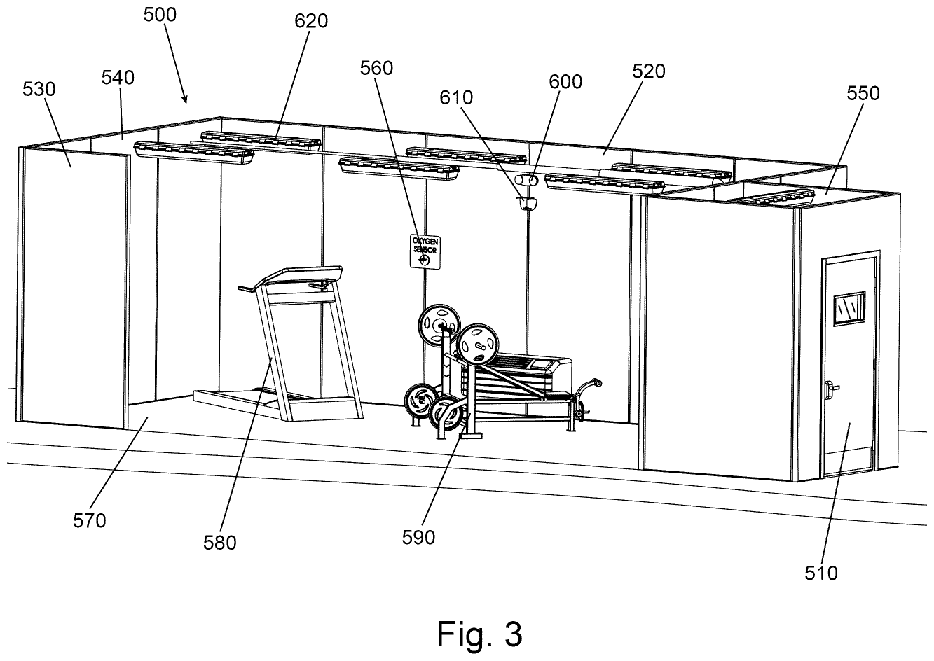

FIG. 3 shows another potentially preferred exercise facility 500 in relation to the disclosure. The sealable facility 500 includes a ceiling (not illustrated), a floor 570, exterior walls 520, 530,540, a vestibule 550 with an exterior door 510, overhead lights 620, an oxygen sensor 560, a sprinkler 600, and an override switch 610. The oxygen level control components (180, 190, 200, 210 of FIG. 2, for example) provide increased and/or decreased oxygen partial pressures as noted within the disclosure. Such components are hidden from view within FIG. 3 as would be the typical arrangement for such a facility 500. A treadmill 580 and weight machine 590 are shown as two possible devices for utilization by a facility user upon increased oxygen partial pressure levels for undertaking a high oxygen-level workout therewith. All precautions are provided for such machinery (present as merely two examples as any number of devices, whether weights, stationary bikes, rowing machines, and the like, may be utilized in such a situation as one of ordinary skill within this art would clearly understand) to be utilized therein to prevent unwanted sparking, flame generation, etc., due to metal contacting, dropping, etc., as well as electrical voltage, etc., creating sparks, and the like, as well. As noted above, such weights, devices, etc., may be provided with outer coatings and/or protective surfaces to prevent static or other electrical charge generation upon utilization or contact with certain surfaces, at least. With such safety concerns provided, and such an adjustable oxygen level permitted for a user, the disclosed facilities, procedures, systems, and/or methods thus accord a user such a capability without endangering themselves to any appreciable degree, ultimately permitting a novel type of athletic activity to provide beneficial improvements heretofore unexplored within the industry.

Such a novel oxygen-enriched (high oxygen partial pressure) enclosed (and sealable) room provides a novel capacity for an individual (or more than one individual) to undertake a physical exertion activity (or activities) therein to enjoy the benefits of greater intensity of exercise while present therein. Such increased intensity of physical exertion accords surprisingly helpful physiological modifications for said individual leading to increased muscular capacity, increased stamina, weight loss, or other personal enjoyment through greater access to oxygen during such physical activities. Possible benefits of increased physical exertion can include increased circulation, increased strength and muscle mass, improved physical performance and other benefits. The ability to utilize a fully enclosed space within such a room, without the need for more in-depth implements (such as gas masks, and the like, accords significant versatility of range of movement for the user, permitting different physical regimens and actions to be undertaken by a user. Thus, standard gym regimens practiced by one or more persons at a time, as well as sexual intercourse, for that matter (basically any physical activity that such freedom of movement and/or larger area for multiple individual's presence) may be undertaken and has shown great promise of significant advantages for user's overall.

It should be understood that various modifications within this disclosure's scope can be made by one of ordinary skill in the art without departing from the spirit thereof. Therefore, it is wished that this disclosure be defined by the scope of the appended claims as broadly as the prior art will permit and given the specification if need be.

1. A sealable and enclosable room filled and maintained with an oxygen-enriched atmosphere through controlled operation of oxygen concentration therein at a partial pressure of oxygen gas greater than 21.2 kPa at sea level.

2. A method utilizing said sealable and enclosable room of claim 1 by at least one individual, said method including the undertaking of physical exertion activity while present therein said room with an oxygen enriched atmosphere of at least said partial pressure level at sea level.

3. The method of claim 2 including physical exertion by at least two people individually or in tandem within said sealable and enclosable room.

4. The sealable and enclosable room of claim 1 comprising a device for controlled delivery of an oxygen-enriched atmosphere exhibiting said partial pressure greater than 21.2 kPa at sea level, wherein said oxygen-enriched atmosphere is controlled through manual operation or automatically through oxygen sensor-controlled operation.

5. The room according to claim 1 which also comprises exercise equipment.

6. The room according to claim 1 comprising at least two manners of ingress/egress.

7. A room according to claim 1 in which the partial pressure of oxygen is more than 25 kPa and less than 100 kPa.

8. A system for delivering and controlling an oxygen-enriched atmosphere within an exercise room suitable for exercise comprising:
an oxygen concentrator;
a valve to control the flow of oxygen from the oxygen concentrator to said exercise room;
a distribution system, to distribute oxygen to various parts of said exercise room;
at least one door or other means for entering and exiting said exercise room; and
at least one sensor in said exercise room to determine the oxygen level therein, wherein the partial pressure of oxygen in said exercise room is controlled to be more than 25 kPa and less than 200 kPa.

9. The system of claim 8 wherein said oxygen concentrator utilizes a pressure swing adsorption process for production of a stream of oxygen rich gas and a stream of nitrogen rich gas.

10. The system of claim 9 wherein said valve to control the flow of oxygen from the oxygen concentrator to the subject room also includes a valve to control the flow of nitrogen from the oxygen concentrator to the subject room.

11. The system of claim 8 wherein said distribution system comprises a system to mix the oxygen with the other gases within said exercise room.

12. The system of claim 8 wherein said at least one door or other means for entering and exiting the room exhibits adequate sealing to prevent excessive leakage of oxygen or entrance of outside air.

13. The system of claim 8 wherein multiple sensors to determine the oxygen level within said exercise room are present.

14. A process for undertaking exercise or other physical exertion activity within the exercise room provided within the system of claim 8, comprising the steps of:
providing said exercise room;
sealing said exercise room to prevent gas ingress and egress upon sealing;
operating said oxygen concentrator to generate said oxygen-enriched atmosphere within said exercise room at a partial pressure of oxygen at a level higher than 21.2 kPa through operation of said valve;
monitoring said at least one an oxygen sensor for ensuring said oxygen-enriched atmosphere is retained within said sealed exercise room; and
undertaking exercise or other physical exertion activity within said sealed oxygen-enriched atmosphere exercise room.
